(19) United States
(12) Patent Application Publication
(10) Pub. No.: US 2009/0320856 A1
(43) Pub. Date: Dec. 31, 2009

(54) Sealant Applicator and Method

(76) Inventors: John Brewer, Marietta, GA (US); Joseph A. Cesa, Cumming, GA (US); Ajay Y. Houde, Duluth, GA (US); Ilona F. Weart, Woodstock, GA (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/164,886

(22) Filed: Jun. 30, 2008

(51) Int. Cl. A61B 19/08; A61M 35/00

(52) U.S. Cl. 128/851; 604/309

(57) ABSTRACT

An applicator and associated method for applying a microbial sealant system and methods for controlling the polymerization of a microbial sealant, the applicator having a housing; a first reservoir element associated with the housing, the first reservoir element containing a polymerization control liquid; a first applicator element adapted to apply a substantially uniform layer polymerization control liquid to the skin; and a second reservoir element integrated with the housing and containing a liquid composed of cyanoacrylate pre-polymers that having polymerization characteristics influenced by the polymerization control liquid, the second reservoir being in fluid communication with a second applicator element adapted to apply a substantially uniform layer of the cyanoacrylate pre-polymers over the layer of polymerization control liquid.

### FIELD OF THE INVENTION

The present invention relates in general to an applicator for applying cyanoacrylate pre-polymers to the skin and associated methods of applying cyanoacrylate pre-polymers to the skin.

### BACKGROUND OF THE INVENTION

Cyanoacrylate polymers have medical uses as an alternative or adjunct to sutures, as a hemostat, to prevent friction blister formation, treating small non-suturable wounds, and in inhibiting surface skin irritation arising from friction between the skin surface and artificial devices such as tapes, prosthetic devices, casts, and the like.

A more recent use of cyanoacrylate polymers is as a surgical drape. This use is generally described at, for example, U.S. Pat. No. 5,807,563 and U.S. Pat. No. 5,730,994, both entitled “Methods for Draping Surgical Incision Sites”.

According to these patents, the in situ formation of a cyanoacrylate polymeric drape at a surgical incision site prior to surgery overcomes many of the problems associated with the use of conventional surgical incise drapes. Such conventional surgical incise drapes are typically pre-formed, sized polymeric films coated with a pressure-sensitive adhesive. After application of an antimicrobial agent such as, for example, surgical prep solution onto the skin surface of the patient, the surgical incise drape is applied, adhesive side down, with sufficient pressure to adhere the drape to the skin. A surgical incision is then made through the drape and surgery is conducted through this incision. After completion of the surgery, the drape is conventionally removed from the skin surface, typically by peeling the drape off the skin.

The most common and potentially serious problem associated with the use of conventional surgical incise drapes is the separation or lifting of the drape from the skin surface during surgery. This problem is related to adhesive failure as well as wrinkling of the pre-formed polymeric film during application. An additional problem associated with pre-formed polymeric films used as surgical incise drapes arises because such drapes do not conform well to the three dimensional contours of the human or other mammalian body thereby increasing the possibility of separation during surgery.

The use of cyanoacrylate polymers as a surgical incise drape provides a significant improvement over conventional surgical incise drapes. In general terms, a layer of cyanoacrylate pre-polymers is applied to the skin, typically after application of an antimicrobial agent such as a surgical site preparation liquid. The layer of cyanoacrylate pre-polymers is allowed to polymerize to form the surgical incise drape

However, uncontrolled polymerization of the cyanoacrylate pre-polymers on the skin may result in undesirable flaking and/or cracking of the cyanoacrylate polymer drape. Uncontrolled polymerization of the cyanoacrylate pre-polymers may also cause a lower level of adhesion to the skin that can result in localized shedding and/or peeling of the cyanoacrylate drape. These phenomena are amplified by the relatively larger surfaces covered by the cyanoacrylate pre-polymers for the in-situ formation of a surgical drape at the incision site in comparison to more typical medical uses of cyanoacrylate polymers to close wounds. Flaking, cracking, shedding and/or peeling compromise the barrier properties of the drape and can reduce the ability of the drape to immobilize microbes by sealing the skin.

In some situations, the surgical site preparation liquids or their residue and/or surgical scrub compositions or soap compositions or their residue may accelerate the cyanoacrylate pre-polymer polymerization reaction such that much shorter polymer chains are generated thereby resulting in a weaker polymer film and/or reduced adhesion to the skin. In other situations, the surgical site preparation liquids or their residue may inhibit the cyanoacrylate pre-polymer polymerization reaction thereby reducing adhesion to the skin or resulting in unsatisfactory clinical drying times.

Accordingly, there is an unmet need for a method of controlling the polymerization of a cyanoacrylate polymeric drape or microbial sealant to reduce or eliminate undesirable flaking, cracking, shedding and/or peeling of the resulting polymeric film on the skin. This is also an unmet need for a convenient and practical method of applying a microbial sealant such that the polymerization of the microbial sealant can be controlled. Moreover, there is an unmet need for an applicator that can be used to apply a microbial sealant such that the polymerization of the microbial sealant can be controlled.

### BRIEF SUMMARY OF THE INVENTION

The problems described above are addressed by the present invention which encompasses an applicator for a microbial sealant system.

The applicator is adapted to apply a microbial sealant system composed of a polymerization control liquid and cyanoacrylate pre-polymers. Desirably, the applicator is adapted to apply the liquids sequentially. The applicator includes a housing having a first end and a second end and a first reservoir associated with the housing. This first reservoir contains a polymerization control liquid. This first reservoir is integrated with the housing. The applicator further includes a first applicator element in fluid communication with the first reservoir.

According to the invention, this first applicator element is adapted to apply a substantially uniform layer of the polymerization control liquid to the skin. In one embodiment, the first applicator element is a spray applicator that deposits a spray of polymerization control liquid on the skin. In another embodiment, the first applicator element includes an applicator head that contacts the skin to deposit polymerization control liquid on the skin. The applicator head may be a liquid-permeable cellular structure such as, for example, a porous sponge material or porous foam material. In yet another embodiment, the applicator element may be a wipe that is pre-saturated with polymerization control liquid and contained in a first reservoir in the form of an impervious flexible package having an opening means. The opening means may be a score, tear strip, re-sealable interlocking fastener or the like.

The applicator includes a second reservoir integrated with the housing. This second reservoir contains liquid cyanoacrylate pre-polymers having polymerization characteristics that are influenced by the polymerization control liquid. This second reservoir is in fluid communication with a second applicator element that is adapted to apply a substantially uniform layer of cyanoacrylate pre-polymers over the layer of polymerization control liquid. Desirably, this second applicator element is an applicator head that contacts the skin to deposit cyanoacrylate pre-polymers over the layer of polymerization control liquid. The applicator head may be a liquid-permeable cellular structure such as, for example, a porous sponge material or porous foam material.

According to an embodiment of the invention, the applicator may be adapted to apply a particular ratio of polymerization control liquid to liquid cyanoacrylate pre-polymers. For example, the applicator may be adapted to apply a ratio of polymerization control liquid to liquid cyanoacrylate-pre-polymers ranging from about 0.125:1 to about 2:1, by weight. As another example, the applicator may be adapted to apply a ratio of polymerization control liquid to liquid cyanoacrylate-pre-polymers ranging from about 0:1 to about 1:1, by weight.

According to the invention, the polymerization control liquid is an aqueous liquid. Desirably, the polymerization control liquid is water. Even more desirably, the polymerization control is sterile water. Other materials or ingredients may be combined or mixed with water including, but not limited to antimicrobial ingredients, polymerization accelerants or polymerization inhibitors.

The liquid cyanoacrylate pre-polymers may be polymerizable formulations composed of cyanoacrylate monomers or polymerizable oligomers. While various cyanoacrylate esters may be used, the cyanoacrylate ester desirably is n-butyl-2-cyanoacrylate. Other cyanoacrylate esters may be used including such esters in which the alkyl group has from 2 to 10 carbon atoms including ethyl, n-propyl, iso-propyl, n-butyl, isobutyl, sec-butyl, n-pentyl, iso-pentyl, n-hexyl, iso-hexyl, 2-ethylhexyl, n-heptyl, octyl, nonyl, and decyl. Mixtures of such compounds can also be used.

Another aspect of the present invention encompasses a method for applying a microbial sealant system. The method includes the steps of: a) applying a substantially uniform layer of a polymerization control liquid to a skin surface; b) applying a substantially uniform layer of liquid cyanoacrylate pre-polymers over the polymerization control liquid; and c) controlling the polymerization of the cyanoacrylate pre-polymers such that longer polymer chains are generated than would be obtained under identical conditions in the absence of the polymerization control liquid thereby forming a skin sealing polymeric film adhered to the skin.

The method may be practiced by applying an amount of polymerization control liquid that is generally proportional to an amount of liquid cyanoacrylate pre-polymers to control the polymerization of the cyanoacrylate pre-polymers. For example, the ratio of polymerization control liquid to liquid cyanoacrylate-pre-polymers may range from about 0.125:1 to about 2:1, by weight. As another example, the ratio of polymerization control liquid to liquid cyanoacrylate-pre-polymers may range from about 0:1 to about 1:1, by weight.

According to the invention, the polymerization control liquid may be applied directly to a skin surface. Alternatively, the polymerization control liquid may be applied to a skin surface already containing or covered by a layer of a surgical site preparation liquid or other medical liquid(s). Typically, such liquid(s) is allowed to dry prior to application of the polymerization control liquid.

The method may be practiced utilizing an applicator that applies generally uniform layers of the polymerization control liquid and the liquid cyanoacrylate pre-polymers sequentially. In an embodiment, the polymerization control liquid may be deposited by spraying it onto the skin surface. In another embodiment, the polymerization control liquid may be deposited by contacting an applicator head onto the skin surface. The applicator head is desirably a liquid-permeable cellular structure such as, for example, a porous sponge material or porous foam material. In yet another embodiment, the polymerization control liquid may be deposited by utilizing a wipe that is pre-saturated with polymerization control liquid. According to the invention, the liquid cyanoacrylate pre-polymer is applied over the polymerization control liquid and may be deposited by contacting an applicator head onto the skin surface containing the polymerization control liquid. The applicator head is desirably a liquid-permeable cellular structure such as, for example, a porous sponge material or porous foam material. Other methods of applying the liquid cyanoacrylate pre-polymer are contemplated including, for example, spraying the liquid.

Yet another aspect of the invention encompasses a method for controlling the polymerization of a microbial sealant. This method includes the steps of: a) applying a substantially uniform layer of a surgical site preparation liquid to a skin surface, the surgical site preparation liquid having a pH that is relatively basic or having ingredients (including, for example, soap residue) that accelerate the rate of cyanoacrylate polymerization; b) applying a substantially uniform layer of a polymerization control liquid to a skin surface, the polymerization control liquid generally lowering the pH at the skin surface to about neutral or minimizing the impact of the cyanoacrylate polymerization rate accelerating ingredients; and c) applying a substantially uniform layer of liquid cyanoacrylate pre-polymers over the polymerization control liquid such that the cyanoacrylate pre-polymers polymerize to generate longer polymer chains than would be obtained under identical conditions in the absence of the polymerization control liquid thereby forming a skin sealing solid polymeric film adhered to the skin

The method may be practiced by applying an amount of polymerization control liquid that is generally proportional to an amount of liquid cyanoacrylate pre-polymers to control the polymerization of the cyanoacrylate pre-polymers. For example, the ratio of polymerization control liquid to liquid cyanoacrylate-pre-polymers may range from about 0.125:1 to about 2:1, by weight. As another example, the ratio of polymerization control liquid to liquid cyanoacrylate-pre-polymers may range from about 0:1 to about 1:1, by weight.

According to the invention, the polymerization control liquid is applied to a skin surface already containing or covered by a layer of a surgical site preparation liquid or other medical liquid(s). Typically, the liquid(s) is allowed to dry before application of the polymerization control liquid.

Another aspect of the invention encompasses a different method for controlling the polymerization of a microbial sealant. The method includes the steps of: a) applying a substantially uniform layer of a surgical site preparation liquid to a skin surface, the surgical site preparation liquid having a pH that is relatively acidic or having ingredients that inhibit the rate of cyanoacrylate polymerization; b) applying a substantially uniform layer of a polymerization control liquid to a skin surface, the polymerization control liquid generally increasing the pH at the skin surface to about neutral or minimizing the impact of the cyanoacrylate polymerization rate inhibiting ingredients; and c) applying a substantially uniform layer of a liquid comprising cyanoacrylate pre-polymers over the polymerization control liquid, such that the cyanoacrylate pre-polymers polymerize more rapidly than under identical conditions in the absence of the polymerization control liquid thereby forming a skin sealing solid polymeric film adhered to the skin.

These and other features and advantages of the invention will become more apparent to one skilled in the art from the following description and claims when read in light of the accompanying drawings.

### BRIEF DESCRIPTION OF THE DRAWINGS

The present invention will be better understood by reading the Detailed Description of the Invention with reference to the accompanying drawing figures, in which like reference numerals denote similar structure and refer to like elements throughout, and in which:

FIG. 1 is a cross-sectional illustration of an exemplary embodiment of an applicator for a microbial sealant system;

FIG. 2 is a perspective illustration of another exemplary embodiment of an applicator for a microbial sealant system;

FIG. 3 is a perspective illustration of yet another exemplary embodiment of an applicator for a microbial sealant system;

FIG. 4 is a modified cross-sectional illustration highlighting a feature of an exemplary embodiment of an applicator for a microbial sealant system.

### DEFINITIONS

The term “liquid cyanoacrylate pre-polymers” refers to polymerizable cyanoacrylate esters in the form of cyanoacrylate monomers or polymerizable oligomers. These polymerizable cyanoacrylate esters are referred to herein as pre-polymers and compositions or formulations containing such esters are also referred to as pre-polymers. Polymerizable cyanoacrylate esters are known in the art and are described in, for example, U.S. Pat. Nos. 3,527,224; 3,591,676; 3,667,472; 3,995,641; 4,035,334; and 4,650,826 the disclosures of each are incorporated herein by reference in their entirety. While various cyanoacrylate esters may be used, the cyanoacrylate ester desirably is n-butyl-2-cyanoacrylate. Other cyanoacrylate esters may be used including such esters in which the alkyl group has from 2 to 10 carbon atoms including ethyl, n-propyl, iso-propyl, n-butyl, isobutyl, sec-butyl, n-pentyl, iso-pentyl, n-hexyl, iso-hexyl, 2-ethylhexyl, n-heptyl, octyl, nonyl, and decyl. Mixtures of such compounds can also be used. In addition, various plasticizers and other formulating aids may be included.

The term “polymerization control liquid” refers to a liquid that is adapted to modify the polymerization rate of a liquid cyanoacrylate pre-polymers on the surface of the skin. Desirably, a single polymerization control liquid may be used to control polymerization when basic or acidic conditions are encountered on the skin surface. The polymerization control liquid should desirably shield, separate and/or buffer the cyanoacrylate polymerization reaction from the catalyzing or inhibiting effects of the materials such as, for example, the substantially dry residue of surgical preparation liquids present on the skin thereby providing a polymerization time in a range of from about thirty (30) seconds to about one (1) minute in comparison to conditions without a layer of the polymerization control liquid. The polymerization control liquid is generally an aqueous liquid having a neutral pH. Exemplary polymerization control liquids include water, including deionized water, sterile water, distilled water and mixtures of water and low molecular weight alcohols (i.e., C1 to C4 alcohols). Minor amounts of antimicrobial materials, polymerization accelerators and/or inhibitors may be included in the aqueous liquid.

The term “microbial sealant” refers to sterile film-forming liquid cyanoacrylate pre-polymer based products that are intended to be applied on the skin over commonly used surgical skin preparation products prior to a surgical incision. Upon polymerization, the microbial sealant bonds to the skin and immobilizes the bacteria which survive the application of antimicrobial surgical skin preparation products. Generally speaking, microbial sealants intended to remain on the skin following the completion of the surgical procedure without requiring removal. The incision is closed and dressed according to existing standards of care and, following surgery, the microbial sealant naturally sloughs off the skin over the course of a few days (e.g., from about two (2) days to about seven (7) days).

### DETAILED DESCRIPTION OF INVENTION

In describing the various embodiments of the present invention, as illustrated in the figures and/or described herein, specific terminology is employed for the sake of clarity. The invention, however, is not intended to be limited to the specific terminology so selected, and it is to be understood that each specific element includes all technical equivalents that operate in a similar manner to accomplish similar functions.

Thus, exemplary embodiments of the invention are presented herein; however, the invention may be embodied in a variety of alternative forms, as will be apparent to those skilled in the art. To facilitate understanding of the invention, and provide a basis for the claims, various figures are included in the description. The figures are not drawn to scale and related elements may be omitted so as to emphasize the novel features of the invention. Structural and functional details depicted in the figures are provided for the purpose of teaching the practice of the invention to those skilled in the art and are not intended to be considered limitations. Directional terms such as left, right, front or rear are provided to assist in the understanding of the invention and are not intended to be considered as limitations.

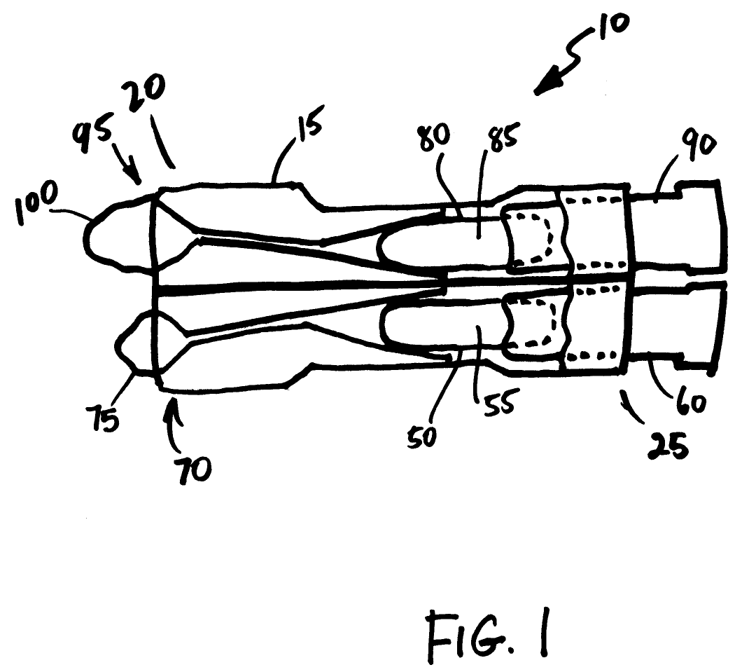

Referring now to FIG. 1, there is shown a cross-sectional illustration of one embodiment of an exemplary applicator 10 for a microbial sealant system. The applicator 10 is adapted to apply a microbial sealant system composed of at least two separated liquid components. These components include a polymerization control liquid and cyanoacrylate pre-polymers.

Generally speaking, the applicator 10 includes a housing 15 having a first end 20 and a second end 25. The housing may be constructed of plastic, wood, metal or other conventional materials.

A first reservoir 50 is associated with the housing 15. This first reservoir 50 contains a polymerization control liquid 55. This first reservoir 50 may be integrated with the housing 15 as shown in FIG. 1. The reservoir may be a sealed flexible tube, packet, pouch or other flexible structure to hold the polymerization control liquid. Alternatively, the first reservoir 50 may be a frangible structure such as a glass vial. In either case, the contents of the first reservoir 50 may be released by activating a release element 60 that releases the polymerization control liquid from the reservoir by an action such as, for example, puncturing the reservoir, compressing the reservoir, tearing the reservoir, or breaking the reservoir. While only a first reservoir 50 is shown, it is contemplated that multiple reservoirs may be used to contain the polymerization control liquid. For example, two or more sealed flexible tubes, packets, pouches or other flexible structures or combinations thereof may be used to contain the polymerization control liquid. Alternatively, multiple frangible structures such as a glass vials or combinations of frangible structures and flexible structures may be used to contain the polymerization control liquid. These multiple reservoirs containing the polymerization control liquid may be arranged to be activated by a single release element 60 or each reservoir containing the polymerization control liquid may be activated by a separate release element 60.

The applicator 10 further includes a first applicator element 70 in fluid communication with the first reservoir 50. This first applicator element 70 includes an applicator head 75 that contacts the skin to deposit polymerization control liquid on the skin. The applicator head 75 may be a liquid-permeable cellular structure such as, for example, a porous sponge material or porous foam material. Alternatively and/or additionally, the applicator head 75 may include a layer of or be entirely composed of a porous non-woven material such as, for example, melt-blown nonwoven fabric, spun-bonded nonwoven fabric or combinations thereof. If multiple reservoirs are used to contain the polymerization control liquid, the multiple reservoirs may be in liquid communication with a single applicator element 70 and applicator head 75.

Alternatively, it is contemplated that each reservoir containing the polymerization control liquid may be in liquid communication with a separate applicator element and applicator head.

Of course, other configurations are contemplated. The key requirement is that the applicator element be adapted to apply a substantially uniform layer of the polymerization control liquid to the skin. For example, the first applicator element 70 may be a conventional roller ball or cylinder roll that transfers polymerization control liquid from the first reservoir 50 to the skin.

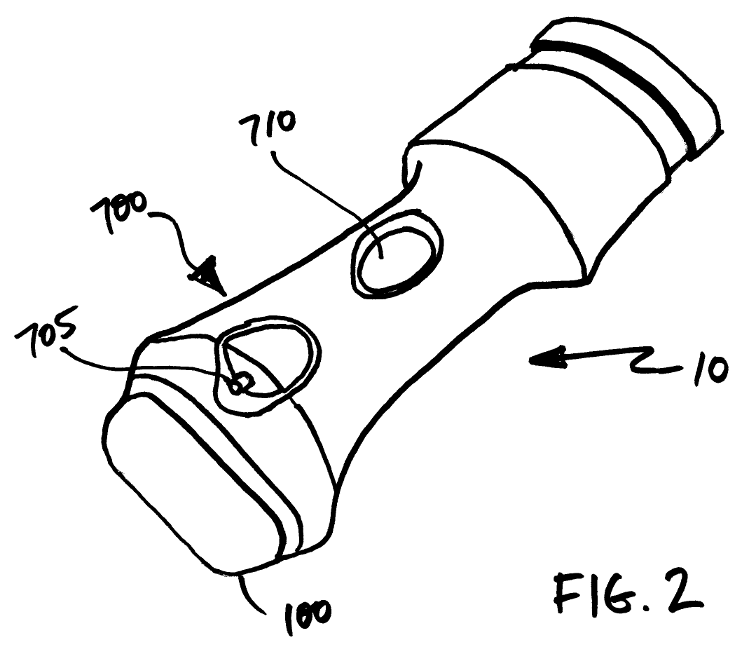

As an example and now referring to FIG. 2, the first applicator element 70 may be spray applicator element 700 formed by a combination of a spray nozzle 705 and a pump bellows having a pressure point 710 that is depressed one or more times to build up pressure to generate a spray of polymerization control liquid on the skin. In another embodiment, the spray applicator element 700 may be formed by a combination of a spray nozzle 705 in fluid communication with a pressurized canister (not shown) that is activated by depressing a pressure point (for example, the pressure point 710). It is contemplated that the spray nozzle 705 itself may be the element that is depressed to released the pressurized polymerization control liquid from the pressurized canister in a manner similar to a conventional aerosol spray can.

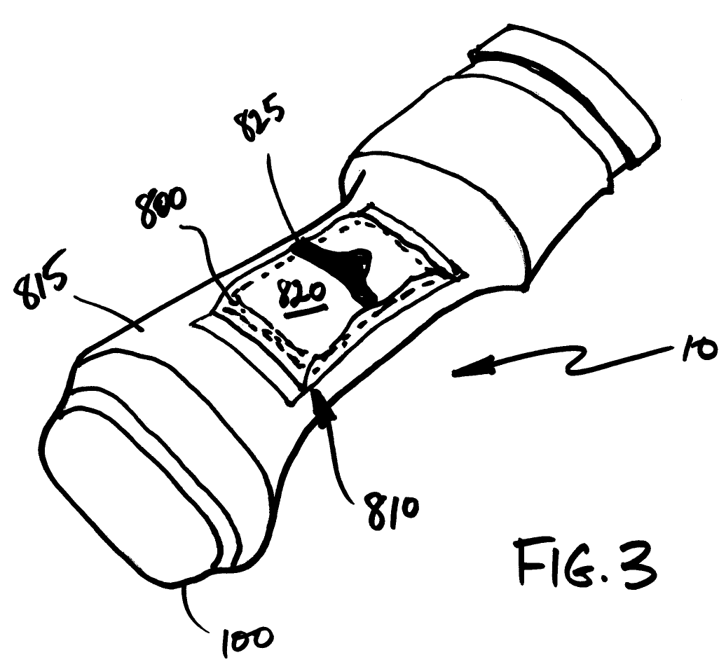

As yet another example and now referring to FIG. 3, the first applicator element 70 may be a wipe 800 that is pre-saturated with polymerization control liquid and contained in a first reservoir 810 associated with the housing 815 in the form of an impervious flexible package 820 having an opening means 825. The opening means may be a score, tear strip, conventional re-sealable interlocking fastener such as, for example, a zip-lock fastener or the like. In use, the impervious flexible package 820 is opened and the wipe 800 that is pre-saturated with polymerization control fluid is used to apply a uniform layer of the polymerization control liquid to the skin.

Referring again to FIG. 1, the applicator 10 includes a second reservoir 80 integrated with the housing 15. This second reservoir 80 contains liquid cyanoacrylate pre-polymers 85 having polymerization characteristics that are influenced by the polymerization control liquid. The second reservoir may be a sealed flexible tube, packet, pouch or other flexible structure to hold the liquid cyanoacrylate pre-polymers. Desirably, the second reservoir 80 may be a frangible structure such as a glass vial. In either case, the contents of the second reservoir 80 may be released by activating a second release element 90 that releases the liquid cyanoacrylate pre-polymers 85 from the reservoir 80 by an action such as, for example, puncturing the reservoir, compressing the reservoir, tearing the reservoir, or breaking the reservoir. While only one second reservoir 80 is shown, it is contemplated that multiple reservoirs may be used to contain the liquid cyanoacrylate pre-polymers. For example, two or more sealed flexible tubes, packets, pouches or other flexible structures or combinations thereof may be used to contain the liquid cyanoacrylate pre-polymers. Alternatively, multiple frangible structures such as a glass vials or combinations of frangible structures and flexible structures may be used to contain the liquid cyanoacrylate pre-polymers. These multiple reservoirs containing the liquid cyanoacrylate pre-polymers may be arranged to be activated by a single second release element or each reservoir containing the liquid cyanoacrylate pre-polymers may be activated by a separate release element. In embodiments of the invention, a single release element 60 may be utilized to active the one or more first reservoirs 50 containing the polymerization control liquid 55 and the one or more second reservoirs 80, containing the liquid cyanoacrylate pre-polymers 85.

This second reservoir 80 is in fluid communication with a second applicator element 95 that is adapted to apply a substantially uniform layer of cyanoacrylate pre-polymers over the layer of polymerization control liquid. Desirably, this second applicator element 95 is an applicator head 100 that contacts the skin to deposit cyanoacrylate pre-polymers 85 over the layer of polymerization control liquid. The applicator head 100 may be a liquid-permeable cellular structure such as, for example, a porous sponge material or porous foam material. Alternatively and/or additionally, the applicator head 100 may include a layer of or be entirely composed of a porous non-woven material such as, for example, melt-blown nonwoven fabric, spun-bonded nonwoven fabric or combinations thereof. If multiple reservoirs are used to contain the cyanoacrylate pre-polymers, the multiple reservoirs may be in liquid communication with a single second applicator element 95 and applicator head 100. Alternatively, it is contemplated that each reservoir containing the cyanoacrylate pre-polymers may be in liquid communication with a separate applicator element and applicator head.

Of course, other configurations are contemplated. The key requirement is that the applicator element be adapted to apply a substantially uniform layer of the liquid cyanoacrylate pre-polymers to the skin. For example, the applicator element 70 may be a conventional roller ball or cylinder roll that transfers polymerization control liquid from the first reservoir 50 to the skin.

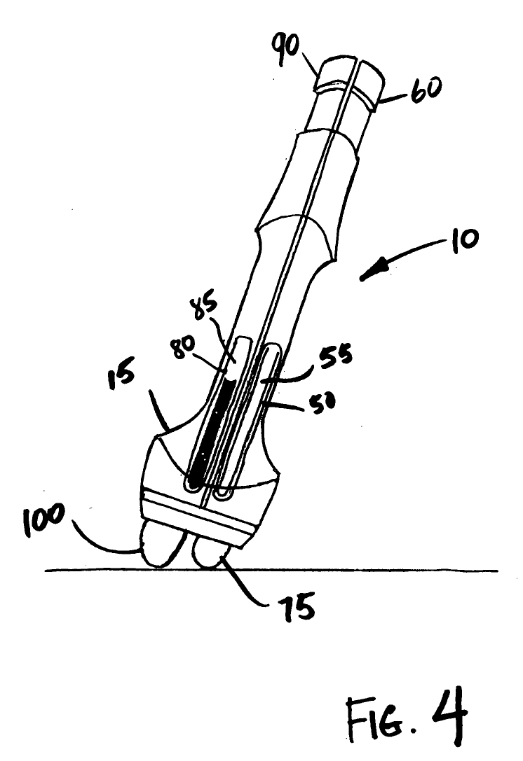

Referring to FIG. 4, the height of the first applicator head 75 for applying the polymerization control liquid may be different from the height of the second applicator head 100 for applying the liquid cyanoacrylate pre-polymers over the polymerization control liquid. This difference in height may be used to create a sidedness to the applicator 10 that requires a user to orient the applicator 10 in a specific manner to more likely result in sequential application of the components of the microbial sealant system. For example, the first applicator head 75 may be lower and the second applicator head 95 may be higher and the corresponding sides of the housing 15 may each have a specific coloration or indicia such that a user will more readily follow instructions to bring the applicator across the skin in a manner that applies the polymerization control liquid before the liquid cyanoacrylate pre-polymers.

According to an embodiment of the invention, the applicator may be adapted to apply a particular ratio of polymerization control liquid to liquid cyanoacrylate pre-polymers. For example, the applicator may be adapted to apply a ratio of polymerization control liquid to liquid cyanoacrylate-pre-polymers ranging from about 0.125:1 to about 2:1, by weight. As another example, the applicator may be adapted to apply a ratio of polymerization control liquid to liquid cyanoacrylate-pre-polymers ranging from about 0.5:1 to about 1:1, by weight.

According to the invention, the polymerization control liquid is an aqueous liquid that is able to function as a weak acid and a weak base. Desirably, the polymerization control liquid is water. Even more desirably, the polymerization control is sterile, deionized water. Other materials or ingredients may be combined or mixed with water including, but not limited to low molecular weight alcohols (C1 to C4), such as, for example, methanol, ethanol, and isopropyl alcohol. For example, an aqueous solution containing from about five (5) percent up to about seventy (70) percent, by weight, isopropyl alcohol may be used. These low molecular weight alcohols provide antimicrobial properties. It is contemplated that other antimicrobial ingredients may be added. In embodiments of the invention, very small amounts of polymerization accelerators and/or inhibitors may also be added to the polymerization control liquid. Exemplary polymerization accelerators may be selected from weak bases such as, for example, ammonia, tri-methyl ammonia, pyridine and ammonium hydroxide. Exemplary polymerization inhibitors may be selected from weak acids such as, for example, acetic acid, formic acid and tri-chloro acetic acid. Depending on the purity of the cyanoacrylate pre-polymer, these polymerization accelerators or inhibitors can be added at the parts per million level. That is, the polymerization accelerators or inhibitors can be added at levels of one (1) milligram of polymerization accelerator per one (1) kilogram of water.

The liquid cyanoacrylate pre-polymers may be polymerizable formulations composed of cyanoacrylate monomers or polymerizable oligomers. While various cyanoacrylate esters may be used, the cyanoacrylate ester desirably is n-butyl-2-cyanoacrylate. Other cyanoacrylate esters may be used including such esters in which the alkyl group has from 2 to 10 carbon atoms including ethyl, n-propyl, iso-propyl, n-butyl, isobutyl, sec-butyl, n-pentyl, iso-pentyl, n-hexyl, iso-hexyl, 2-ethylhexyl, n-heptyl, octyl, nonyl, and decyl. Mixtures of such compounds can also be used. The liquid cyanoacrylate pre-polymers may contain additives such as plasticizing agents to improve film flexibility and conformance, viscosity modifiers to aid in application of the liquid composition, free radical and anionic scavengers to stabilize the product prior to use, biocidal agents to kill immobilized bacteria under the film, and the like.

The present invention encompasses a method for applying a microbial sealant system. The microbial sealant system includes at least two components. The components are a polymerization control liquid and liquid cyanoacrylate pre-polymers. According to the invention, the method includes the steps of: a) applying a substantially uniform layer of a polymerization control liquid to a skin surface; b) applying a substantially uniform layer of liquid cyanoacrylate pre-polymers over the polymerization control liquid; and c) controlling the polymerization of the cyanoacrylate pre-polymers such that longer polymer chains are generated than would be obtained under identical conditions in the absence of the polymerization control liquid thereby forming a skin sealing polymeric film adhered to the skin. Generally speaking, the liquid cyanoacrylate pre-polymers are applied immediately after the polymerization control liquid is applied.

The method may be practiced by applying an amount of polymerization control liquid that is generally proportional to an amount of liquid cyanoacrylate pre-polymers to control the polymerization of the cyanoacrylate pre-polymers. For example, the ratio of polymerization control liquid to liquid cyanoacrylate-pre-polymers may range from about 0.125:1 to about 2:1, by weight. As another example, the ratio of polymerization control liquid to liquid cyanoacrylate-pre-polymers may range from about 0.5:1 to about 1:1, by weight.

According to the invention, the polymerization control liquid may be applied directly to a skin surface. However, in most situations, the polymerization control liquid would be applied to a skin surface already containing or covered by a layer of a surgical site preparation liquid or other medical liquid(s). Generally speaking, the surgical site preparation liquid is allowed to dry prior to applying the polymerization control liquid.

The method may be practiced utilizing an applicator that applies generally uniform layers of the polymerization control liquid and the liquid cyanoacrylate pre-polymers sequentially. In an embodiment, the polymerization control liquid may be deposited by spraying it onto the skin surface. In another embodiment, the polymerization control liquid may be deposited by contacting an applicator head onto the skin surface. The applicator head is desirably a liquid-permeable cellular structure such as, for example, a porous sponge material or porous foam material. In yet another embodiment, the polymerization control liquid may be deposited by utilizing a wipe that is pre-saturated with polymerization control liquid. According to the invention, the liquid cyanoacrylate pre-polymer is applied over the polymerization control liquid and may be deposited by contacting an applicator head onto the skin surface containing the polymerization control liquid. The applicator head is desirably a liquid-permeable cellular structure such as, for example, a porous sponge material or porous foam material. Other methods of applying the liquid cyanoacrylate pre-polymer are contemplated including, for example, spraying the liquid.

While the inventors should not be held to a particular theory of operation, it is generally thought that certain types of commonly used surgical site preparation liquids or their residue, and/or surgical scrub preparations and/or soaps or their residues, particularly such materials having a basic pH or ingredients such as amines or alkali ions, may accelerate the cyanoacrylate pre-polymer polymerization reaction such that much shorter polymer chains are generated thereby resulting in a weaker polymer film and/or reduced adhesion to the skin. Examples of these surgical site preparation liquids include formulations containing quaternary amines or benzalkonium chloride. The presence of catalysts or accelerators such as alcohols, amines, ammonia, sodium hydroxide, alkali ions from soap residue and the like, is thought to cause polymerization to occur with such extreme rapidity that weak bonds are formed. In some situations, the surgical site preparation liquids and/or residue can be taken up into the applicator head for the cyanoacrylate pre-polymer such that it accelerates polymerization in the applicator head to block or shut off the flow of liquid cyanoacrylate pre-polymer and interfere with dispensing and/or prevent full dispensing of the cyanoacrylate pre-polymer.

In other situations, it is generally thought that other types of commonly used surgical site preparation liquids, particularly such liquids having an acid pH, may inhibit the cyanoacrylate pre-polymer polymerization reaction thereby extending the polymerization time such that health care professionals may contact the un-polymerized cyanoacrylate microbial sealant thereby compromising the barrier properties. Alternatively and/or additionally, the polymerization time (also called “drying time”) for the cyanoacrylate microbial sealant may be so long as to result in clinically unsatisfactory drying times and/or reduced adhesion to the skin. Examples of these surgical site preparation liquid include povidone-iodine based formulations. For example, BETADINE® Solution has a pH in the range of about 4.5 to about 5.

Uncontrolled polymerization of the cyanoacrylate pre-polymers on the skin is thought to be a source of undesirable flaking and/or cracking of the cyanoacrylate polymer drape. Additionally, uncontrolled polymerization of the cyanoacrylate pre-polymers is also thought to be an important factor in lowering the level of adhesion to the skin that can result in localized shedding and/or peeling of the cyanoacrylate drape. These phenomena are amplified by the relatively larger surfaces covered by the cyanoacrylate pre-polymers for the in-situ formation of a surgical drape at the incision site in comparison to more typical medical uses of cyanoacrylate polymers to close wounds. Flaking, cracking, shedding and/or peeling compromise the barrier properties of the drape and can reduce the ability of the drape to immobilize microbes by sealing the skin.

Generally speaking, polymerization of the liquid cyanoacrylate pre-polymers ranges from less than thirty (30) seconds to more than ten (10) minutes. Polymerization times much less than thirty (30) seconds are generally thought to be undesirable because too rapid polymerization results in short polymer chains that are associated with undesirable phenomena noted above. Alternatively and/or additionally, polymerization times much less than thirty (30) seconds may not provide enough time to apply the liquid cyanoacrylate pre-polymers to the desired area. However, polymerization times much longer than one (1) minute are also generally thought to be undesirable because of the increase likelihood of contact by healthcare professionals with the un-polymerized microbial sealant. Such contact may compromise the barrier properties of the microbial sealant. Long polymerization times (e.g., several minutes) are typically unsatisfactory in clinical settings because time is frequently a critical factor in procedures where microbial sealants are used. Moreover, polymerization times of several minutes or longer may result in poor adhesion to the skin.

According to the invention, a single polymerization control liquid that can be both a weak acid or a weak base may be used to control polymerization when basic or acidic conditions are encountered on the skin surface. Generally speaking, water works well and, in some situations, water and low molecular weight alcohol mixtures work well. Although the inventors should not be bound to any particular theory of operation, when basic conditions (or other polymerization accelerating conditions) are present on the skin surface, a thin layer of neutral polymerization control liquid is sufficient to shield and/or buffer the polymerization reaction from the catalyzing effects of the basic conditions on the skin thereby slowing the polymerization time in comparison to conditions without the polymerization control liquid. Similarly, when acidic conditions (or other polymerization inhibiting conditions) are present on the skin surface, a thin layer of neutral polymerization control liquid is sufficient to shield and/or buffer the polymerization reaction from the inhibiting effects of the acidic conditions on the skin thereby accelerating the polymerization time in comparison to conditions without the polymerization control liquid.

Yet another aspect of the invention encompasses a method for controlling the polymerization of a microbial sealant. This method includes the steps of: a) applying a substantially uniform layer of a surgical site preparation liquid to a skin surface, the surgical site preparation liquid having a pH that is relatively basic or having ingredients (including, for example, soap residue) that accelerate the rate of cyanoacrylate polymerization; b) applying a substantially uniform layer of a polymerization control liquid to a skin surface, the polymerization control liquid generally lowering the pH at the skin surface to about neutral or minimizing the impact of the cyanoacrylate polymerization rate accelerating ingredients; and c) applying a substantially uniform layer of liquid cyanoacrylate pre-polymers over the polymerization control liquid such that the cyanoacrylate pre-polymers polymerize to generate longer polymer chains than would be obtained under identical conditions in the absence of the polymerization control liquid thereby forming a skin sealing solid polymeric film adhered to the skin

The method may be practiced by applying an amount of polymerization control liquid that is generally proportional to an amount of liquid cyanoacrylate pre-polymers to control the polymerization of the cyanoacrylate pre-polymers. For example, the ratio of polymerization control liquid to liquid cyanoacrylate-pre-polymers may range from about 0.125:1 to about 2:1, by weight. As another example, the ratio of polymerization control liquid to liquid cyanoacrylate-pre-polymers may range from about 0.5:1 to about 1:1, by weight. In an embodiment of this method, the method is practiced by also applying an amount of polymerization control liquid that is generally proportional to an amount of surgical site preparation liquid already present on the skin surface. According to the invention, the polymerization control liquid is applied to a skin surface already containing or covered by a layer of a surgical site preparation liquid (that has been allowed to dry) or other medical liquid(s).

Another aspect of the invention encompasses a different method for controlling the polymerization of a microbial sealant. The method includes the steps of: a) applying a substantially uniform layer of a surgical site preparation liquid to a skin surface, the surgical site preparation liquid having a pH that is relatively acidic or having ingredients that inhibit the rate of cyanoacrylate polymerization; b) applying a substantially uniform layer of a polymerization control liquid to a skin surface, the polymerization control liquid generally increasing the pH at the skin surface to about neutral or minimizing the impact of the cyanoacrylate polymerization rate inhibiting ingredients; and c) applying a substantially uniform layer of a liquid comprising cyanoacrylate pre-polymers over the polymerization control liquid, such that the cyanoacrylate pre-polymers polymerize more rapidly than under identical conditions in the absence of the polymerization control liquid thereby forming a skin sealing solid polymeric film adhered to the skin. For example, BETADINE® Solution has a pH of about 4.5 to 5.5. Bare skin has a pH of about 4.0 to 5.5.

### EXAMPLE

This example illustrates an aspect of the present invention carried out with two commercially available products.

BETADINE® Solution (aqueous solution of 10% povidone-iodine) is a commonly used surgical preparation liquid and is available from Purdue Product, L.P. of Stamford, Conn. Povidone-iodine is a water-soluble complex of iodine with polyvinylpyrrolidone. It is a fast-acting, broad-spectrum antiseptic that kills gram-positive and gram-negative bacteria (including antibiotic resistant organisms), as well as most fungi/yeasts, viruses and protozoa. It is indicated for preoperative skin preparation of patients.

INTEGUSEAL® Microbial Sealant is a sterile film-forming liquid cyanoacrylate pre-polymer based product provided in a ready-to-use applicator. It is available from Kimberly-Clark Health Care of Roswell, Ga. INTEGUSEAL® is intended to be applied on the skin over commonly used surgical skin preparation products prior to a surgical incision. Upon polymerization, INTEGUSEAL® bonds to the skin and immobilizes the bacteria which survive the application of antimicrobial surgical skin preparation products. INTEGUSEAL® can be used in combination with surgical skin preparations including iodophors and 2% chlorhexidine gluconate with alcohol. INTEGUSEAL® is intended to remain on the skin following the completion of the surgical procedure without requiring removal. The incision is closed and dressed according to existing standards of care and, following surgery, INTEGUSEAL® naturally sloughs off the skin over the course of a few days.

Two separate, hairless locations measuring approximately two (2) inches by two (2) inches (approximately 5 cm by 5 cm) on the arm of a test subject were rinsed with water and allowed to dry. BETADINE® Solution was applied to the two separate, utilizing a conventional applicator and allowed to dry. INTEGUSEAL® Microbial Sealant was then applied to one of the test locations directly over the dry BETADINE® Solution and monitored for the amount of time required for polymerization to occur such that the surface of the microbial sealant was dry. Dryness of the INTEGUSEAL® Microbial Sealant was determined by applying a nitrile rubber glove covered finger to the surface of the sealant and observing whether any residue was present on the glove. Different locations were contacted for each determination. That is, the same location was not contacted twice. The time to dry was recorded as the Control.

Distilled water was sprayed from an atomizer onto the second skin substrate over the dry BETADINE® Solution. The amount of the water deposited on the skin from the atomized mist was approximately 0.4 grams (±0.1 gram) which completely covered the dry BETADINE® Solution without pooling or puddling. INTEGUSEAL® Microbial Sealant was immediately applied (less than five (5) seconds after the atomized mist was deposited on the skin) over the area utilizing the conventional INTEGUSEAL® applicator. The amount of INTEGUSEAL® Microbial Sealant applied was approximately 0.5 grams. INTEGUSEAL® Microbial Sealant is a blue/violet-colored, free flowing liquid cyanoacrylate pre-polymer contained in a glass ampoule. The INTEGUSEAL® Microbial Sealant is contained within a glass ampoule or ampoules housed within a nylon applicator. The glass ampoule(s) is broken by pushing the rear of the plastic applicator forward, allowing the INTEGUSEAL® Microbial Sealant to flow to the foam tip of the applicator. INTEGUSEAL® Microbial Sealant is applied to the surgical incision site by pressing the foam tip gently on the skin. Application is similar to painting with a foam pad to deposit a layer of INTEGUSEAL® Microbial Sealant on the skin. The amount of time required for polymerization to occur such that the surface of the microbial sealant was dry was monitored according to the “gloved finger” procedure noted above and recorded.

The first skin model had a polymerization time (to dry surface) of about 1.5 minutes. The second skin model that was misted with water prior to applying the microbial sealant (liquid cyanoacrylate pre-polymer) had a polymerization time (to dry surface) of about 30 seconds. The difference provided by the water mist was approximately ⅓ of the time of the Control.

These and other features and advantages of the invention will become more apparent to one skilled in the art from the following description and claims when read in light of the accompanying drawings.

While particular embodiments of the present invention have been described herein; it will be apparent to those skilled in the art that alterations and modifications may be made to the described embodiments without departing from the scope of the appended claims.

What is claimed is:

1. An applicator for a microbial sealant system, the applicator comprising:
a housing having a first end and a second end;
a first reservoir element associated with the housing, the first reservoir element containing a polymerization control liquid;
a first applicator element adapted to apply a substantially uniform layer of polymerization control liquid to the skin; and
a second reservoir element integrated with the housing and containing a liquid comprising cyanoacrylate pre-polymers that having polymerization characteristics influenced by the polymerization control liquid, the second reservoir being in fluid communication with a second applicator element adapted to apply a substantially uniform layer of the cyanoacrylate pre-polymers over the layer of polymerization control liquid.

2. The applicator of claim 1 wherein the first reservoir element is integrated with the housing and the first applicator is in fluid communication with the first reservoir.

3. The applicator of claim 2 wherein the first applicator element is a spray applicator that deposits a spray of polymerization control liquid on the skin.

4. The applicator of claim 2 wherein the first applicator element is an applicator head that contacts the skin to deposit polymerization control liquid on the skin.

5. The applicator of claim 1 wherein the first applicator element is a wipe that is pre-saturated with polymerization control liquid and contained in a first reservoir element comprising an impervious flexible package having an opening means.

6. The applicator of claim 1 wherein the second applicator element is a liquid-permeable cellular structure.

7. The applicator of claim 1 wherein the applicator is adapted to apply a ratio of polymerization control liquid to liquid cyanoacrylate-pre-polymers ranging from about 0.125:1 to about 2:1, by weight.

8. The applicator of claim 1 wherein the polymerization control liquid is selected from deionized water and mixtures of deionized water and low molecular weight alcohols.

9. The applicator of claim 1 wherein the liquid cyanoacrylate pre-polymers are selected from cyanoacrylate esters in which the alkyl group has from 2 to 10 carbon atoms including ethyl, n-propyl, iso-propyl, n-butyl, isobutyl, sec-butyl, n-pentyl, iso-pentyl, n-hexyl, iso-hexyl, 2-ethylhexyl, n-heptyl, octyl, nonyl, and decyl and mixtures thereof.

10. A method for applying a microbial sealant system, the method comprising:
applying a substantially uniform layer of a polymerization control liquid to a skin surface;
applying a substantially uniform layer of a liquid comprising cyanoacrylate pre-polymers over the polymerization control liquid; and
controlling the polymerization of the cyanoacrylate pre-polymers to generate longer polymer chains than would be obtained under identical conditions in the absence of the polymerization control liquid thereby forming a skin sealing solid polymeric film adhered to the skin.

11. The method of claim 10 wherein the polymerization control liquid and cyanoacrylate pre-polymers are applied at a ratio ranging from about 0.125:1 to about 2:1, by weight.

12. The method of claim 10 wherein the polymerization control liquid is applied to a skin surface containing a layer of a surgical site preparation liquid.

13. The method of claim 10 wherein the polymerization control liquid is selected from deionized water and mixtures of deionized water and low molecular weight alcohols.

14. The method of claim 10 wherein the cyanoacrylate pre-polymers are selected from cyanoacrylate esters in which the alkyl group has from 2 to 10 carbon atoms including ethyl, n-propyl, iso-propyl, n-butyl, isobutyl, sec-butyl, n-pentyl, iso-pentyl, n-hexyl, iso-hexyl, 2-ethylhexyl, n-heptyl, octyl, nonyl, and decyl and mixtures thereof.

15. A method for controlling the polymerization of a microbial sealant, the method comprising:
applying a substantially uniform layer of a surgical site preparation liquid to a skin surface, the surgical site preparation liquid having a pH that is relatively basic or having ingredients that accelerate the rate of cyanoacrylate polymerization;
applying a substantially uniform layer of a polymerization control liquid to a skin surface, the polymerization control liquid generally lowering the pH at the skin surface to about neutral or minimizing the impact of the cyanoacrylate polymerization rate accelerating ingredients; and
applying a substantially uniform layer of a liquid comprising cyanoacrylate pre-polymers over the polymerization control liquid,
wherein the cyanoacrylate pre-polymers polymerize to generate longer polymer chains than would be obtained under identical conditions in the absence of the polymerization control liquid thereby forming a skin sealing solid polymeric film adhered to the skin.

16. The method of claim 15 wherein the polymerization control liquid and cyanoacrylate pre-polymers are applied at a ratio ranging from about 0.125:1 to about 2:1, by weight.

17. The method of claim 15 wherein the polymerization control liquid is selected from deionized water and mixtures of deionized water and low molecular weight alcohols.

18. The method of claim 15 wherein the liquid cyanoacrylate pre-polymer is selected from cyanoacrylate esters in which the alkyl group has from 2 to 10 carbon atoms including ethyl, n-propyl, iso-propyl, n-butyl, isobutyl, sec-butyl, n-pentyl, iso-pentyl, n-hexyl, iso-hexyl, 2-ethylhexyl, n-heptyl, octyl, nonyl, and decyl and mixtures thereof.

19. A method for controlling the polymerization of a microbial sealant, the method comprising:
applying a substantially uniform layer of a surgical site preparation liquid to a skin surface, the surgical site preparation liquid having a pH that is relatively acidic or having ingredients that inhibit the rate of cyanoacrylate polymerization;
applying a substantially uniform layer of a polymerization control liquid to a skin surface, the polymerization control liquid generally increasing the pH at the skin surface to about neutral or minimizing the impact of the cyanoacrylate polymerization rate inhibiting ingredients; and
applying a substantially uniform layer of a liquid comprising cyanoacrylate pre-polymers over the polymerization control liquid,
wherein the cyanoacrylate pre-polymers polymerize more rapidly than under identical conditions in the absence of the polymerization control liquid thereby forming a skin sealing solid polymeric film adhered to the skin.
